(19) United States
(12) Patent Application Publication
(10) Pub. No.: US 2022/0313586 A1
(43) Pub. Date: Oct. 6, 2022

(54) COSMETIC COMPOSITION WITH GLAUCINE, RETINOL AND PEPTIDES TO COMBAT SKIN AGING

(71) Applicant: L'OREAL, Paris (FR)

(72) Inventors: Kristin Gilida STEELEY, Branchburg, NJ (US); Nada Hisham BAALBAKI, Hoboken, NJ (US); Stephen Matthew LYNCH, Westfield, NJ (US); Jaimie MECCA, Clifton, NJ (US)

(73) Assignee: L'OREAL, Paris (FR)

(21) Appl. No.: 17/218,960

(22) Filed: Mar. 31, 2021

(51) Int. Cl. A61K 8/64; A61K 8/67; A61K 8/49; A61Q 19/08; A61Q 19/00; A61K 8/11; A61K 8/34; A61K 8/33; A61K 8/73; A61K 8/37; A61K 8/36; A61K 8/55; A61K 8/92; A61K 8/891; A61K 8/81; A61K 8/19; A61K 8/25; A61K 8/46

(52) U.S. Cl. CPC A61K 8/645; A61K 8/64; A61K 8/671; A61K 8/4926; A61Q 19/08; A61Q 19/007; A61K 8/11; A61K 8/345; A61K 8/33; A61K 8/73; A61K 8/375; A61K 8/361; A61K 8/553; A61K 8/922; A61K 8/891; A61K 8/8147; A61K 8/19; A61K 8/25; A61K 8/466; A61K 2800/49; A61K 2800/48; A61K 2800/56; A61K 2800/5922; A61K 2800/524

(57) ABSTRACT

A cosmetic composition to combat skin aging, particularly on the neck, includes a blend of skin actives comprising at least the combination of glaucine, retinol and a blend of peptides. The skin actives are provided in an aqueous carrier in a single phase, biphase or emulsion wherein the cosmetic composition is a leave on treatment in the form of a cream, gel, lotion, toner, or serum. The cosmetic composition provides one or more benefits, including, but not limited to improvement in many signs of skin aging, particularly on the neck, such as lines, wrinkles, crepiness, and sagging, the benefits conferred by one or more of increasing firmness, increasing elasticity, improving hydration, and reducing the presence superficial adipose tissue. The cosmetic composition may include one or more of additional skin actives, solvents, surfactants, oils, thickeners, preservatives, brightening agents, effect pigments, essential oils, plant extracts, or combinations of these.

### FIELD OF THE INVENTION

The present disclosure relates to a water-based cosmetic composition to combat skin aging, particularly on the neck, that includes a blend of skin actives comprising at least the combination of glaucine, retinol and a blend of peptides.

### BACKGROUND OF THE INVENTION

Skin aging and the attendant visible and textural changes to skin are a major focus of a wide variety of cosmetic products. Skin on the face and neck are particularly important targets for such treatments, especially skin on the neck which has different physical characteristics than skin on the face, and consequently shows aging in particular ways, which can include lines, wrinkles, crepiness, and sagging. Among skin cleansers, toners, moisturizers and other forms of treatment, a wide variety of skin care products provide blends of ingredients that address the signs of aging. While many ingredients in these products are known to have relevant biological effects, it remains a challenge to provide cosmetic treatments that include amounts and combinations of such ingredients sufficient to be effective and are also lightweight and pleasant to use and don't create an oily finish or cause dryness or irritation.

Accordingly, there is a need in the art for a cosmetic composition that confers one or more of anti-aging benefits in a formulation that is lightweight and pleasant to use and does not create an oily finish or cause dryness or irritation. In various embodiments, the cosmetic composition according to the instant invention contains a unique combination of active ingredients that work together to reduce the signs of aging on skin, particularly skin in the neck area, including significant improvements in many signs of neck skin aging such as reduction of lines, wrinkles, crepiness, and sagging, with improved firmness and elasticity, increased hydration of tissue, reduced water loss, and reduced presence superficial adipose tissue.

### BRIEF SUMMARY OF THE INVENTION

The summary is provided to introduce a selection of concepts in a simplified form that are further described below in the detailed description of the invention. This summary is not intended to identify key features of the claimed subject matter, nor is it intended to be used as an aid in determining the scope of the claimed subject matter.

In an exemplary embodiment, the invention provides an anti-aging cosmetic composition that include a blend of skin actives comprising at least the combination of glaucine, retinol and a blend of peptides. The blend of skin actives is provided in an aqueous carrier system, and may be in any form such as, but not limited to, a single phase, biphase, or emulsion, wherein the cosmetic composition is a leave on treatment in a product form selected from, but not limited to, a cream, gel, lotion, toner, serum, or combination of these. The cosmetic composition provides one or more benefits, including, but not limited to improvement in many signs of skin aging, particularly on the neck, such as lines, wrinkles, crepiness, and sagging, the benefits conferred by one or more of increasing firmness, increasing elasticity, improving hydration, and reducing the presence superficial adipose tissue.

The cosmetic composition may include one or more of additional skin actives to provide one or more additional benefits, including, but not limited to brightening/whitening, blemish control or reduction, reduction of redness, and hydration.

In some embodiments, the cosmetic composition may include additional ingredients including one or more of each of solvents, surfactants, oils, thickeners, preservatives, brightening agents, effect pigments, essential oils, plant extracts, or combinations of these.

In some embodiments, the skin actives are provided in an aqueous carrier in a single phase, biphase or emulsion wherein the cosmetic composition is a leave on treatment in the form of a cream, gel, lotion, toner, or serum.

In some embodiments, glaucine is present from about 0.001% to about 0.1%, retinol is present from about 0.05% to about 0.5%, tetradecyl aminobutyroylvalylaminobutyric urea trifluoroacetate is present from about 0.002% to about 5% and hydrolyzed rice protein is present from about 0.125% to about 5%, all percentages by weight, based on the total weight of the cosmetic composition.

In some embodiments, glaucine is present from about 0.0025% to about 0.0005%, retinol is present from about 0.1% to about 0.2%, tetradecyl aminobutyroylvalylaminobutyric urea trifluoroacetate is present from about 0.0022% to about 0.00275% and hydrolyzed rice protein is present from about 0.125% to about 0.25%, all percentages by weight, based on the total weight of the cosmetic composition.

In some embodiments, glaucine is present at about 0.005%, retinol is present at about 0.2%, tetradecyl aminobutyroylvalylaminobutyric urea trifluoroacetate is present from about 0.00275% and hydrolyzed rice protein is present at about 0.25%, all percentages by weight, based on the total weight of the cosmetic composition.

In some embodiments, the blend of peptides is present from about 0.001% to about 5%, or from about 0.002% to about 5%.

In some embodiments, the cosmetic composition comprises one or more of:
a. one or more water-soluble solvents comprising Caprylyl Glycol and Glycerin,
b. one or more thickeners comprising xanthan gum,
c. one or more surfactants comprising glyceryl stearate, stearic acid and potassium cetyl phosphate,
d. one or more oils comprising Helianthus Annuus Seed Oil Unsaponifiables, Lavandula Angustifolia Oil, Mentha Piperita Oil, Rosmarinus Officinalis Leaf Oil, Dimethicone, Hydrogenated Polyisobutene, and Myristyl Myristate,
e. one or more filler comprising vinyl dimethicone/methicone silsesquioxane crosspolymer, Ammonium Polyacryloyldimethyl Taurate, Methyl Methacrylate Crosspolymer, and silica silylate, and
f. one or more additional ingredients comprising Capryloyl Glycine as a preservative, and Sodium Hydroxide, or
g. combinations thereof.

In an exemplary embodiment, the invention provides a cosmetic composition comprising:
a. a blend of skin actives comprising at least a glaucine, retinol, and a blend of peptides comprising tetradecyl aminobutyroylvalylaminobutyric urea trifluoroacetate and hydrolyzed rice protein; and
b. an aqueous carrier system comprising water, and wherein the cosmetic composition increases firmness, increases elasticity, increases hydration, reduces the presence superficial adipose tissue, or combinations thereof.

In an exemplary embodiment, the invention provides a cosmetic composition comprising:
c. a blend of skin actives comprising at least a glaucine present from about 0.0025% to about 0.005% by weight, based on the total weight of the cosmetic composition, retinol, and a blend of peptides; and
d. an aqueous carrier system comprising water, and wherein the cosmetic composition increases firmness, increases elasticity, increases hydration, reduces the presence superficial adipose tissue, or combinations thereof.

In an exemplary embodiment, the invention provides a cosmetic composition comprising:
h. a blend of skin actives comprising at least a glaucine, retinol, and a blend of peptides comprising tetradecyl aminobutyroylvalylaminobutyric urea trifluoroacetate and hydrolyzed rice protein; and
i. an aqueous carrier system comprising water,

wherein glaucine is present from about 0.0025% to about 0.005%, retinol is present from about 0.1% to about 0.2%, tetradecyl aminobutyroylvalylaminobutyric urea trifluoroacetate is present from about 0.0022% to about 0.00275% and hydrolyzed rice protein is present from about 0.125% to about 0.25%, all percentages by weight, based on the total weight of the cosmetic composition, and

wherein the aqueous carrier system comprises at least one water-soluble solvent, at least one oil, at least one surfactant, at least one thickener, at least one filler, or combinations thereof, and further comprises one or more of additional skin actives, preservatives, brightening agents, effect pigments, essential oils, plant extracts, or combinations thereof, and

wherein the cosmetic composition is provided as an emulsion and is a leave on treatment in the form of a cream, gel, lotion, toner, or serum, and wherein the cosmetic composition increases firmness, increases elasticity, increases hydration, reduces the presence superficial adipose tissue, or combinations thereof.

In some embodiments, the cosmetic composition comprises water-soluble solvent comprising Caprylyl Glycol and Glycerin, at least one thickener comprising xanthan gum, surfactant comprising glyceryl stearate, stearic acid and potassium cetyl phosphate, oil comprising Helianthus Annuus Seed Oil Unsaponifiables, Lavandula Angustifolia Oil, Mentha Piperita Oil, Rosmarinus Officinalis Leaf Oil, Dimethicone, Hydrogenated Polyisobutene, and Myristyl Myristate, filler comprising vinyl dimethicone/methicone silsesquioxane crosspolymer, Ammonium Polyacryloyldimethyl Taurate, Methyl Methacrylate Crosspolymer, and silica silylate, and additional ingredients comprising Capryloyl Glycine as a preservative, and Sodium Hydroxide.

These and other aspects of the invention are set out in the appended claims and described in greater detail in the detailed description of the invention.

This disclosure describes exemplary embodiments in accordance with the general inventive concepts and is not intended to limit the scope of the invention in any way. Indeed, the invention as described in the specification is broader than and unlimited by the exemplary embodiments set forth herein, and the terms used herein have their full ordinary meaning.

### DETAILED DESCRIPTION OF THE INVENTION

“Keratinous tissue,” as used herein, includes, but is not limited to, skin, hair, and nails.

The disclosure relates to a cosmetic composition to combat skin aging includes a blend of skin actives comprising at least the combination of glaucine, retinol and a blend of peptides. The skin actives are provided in an aqueous carrier in a single phase, biphase or emulsion wherein the cosmetic composition is a leave on treatment in the form of a cream, gel, lotion, toner, or serum.

The cosmetic composition provides one or more benefits, including, but not limited to improvement in many signs of skin aging, particularly on the neck, such as lines, wrinkles, crepiness, and sagging, the benefits conferred by one or more of increasing firmness, increasing elasticity, improving hydration, and reducing the presence superficial adipose tissue.

The cosmetic composition may include one or more of additional skin actives to provide one or more additional benefits, including, but not limited to brightening/whitening, blemish control or reduction, reduction of redness, and hydration. And, the cosmetic composition may include additional ingredients including one or more of each of solvents, surfactants, oils, thickeners, preservatives, brightening agents, effect pigments, essential oils, plant extracts, or combinations of these.

Applicant's inventors recognize that skin on the neck has different physical characteristics than skin on the face, and consequently shows aging in particular ways. The cosmetic composition according to the invention contains a unique combination of three active ingredients, including retinol, particularly, encapsulated retinol, a blend of peptides that include tripeptides, and glaucine that work together to reduce the signs of aging on skin, particularly skin in the neck area.

Applicant's inventors have determined that a blend of skin actives comprising at least the combination of glaucine, retinol and a blend of peptides, as disclosed herein, surprisingly confers one or more benefits of increased firmness, increased elasticity, improved hydration of tissue, reduced water loss, and reduced presence superficial adipose tissue.

Without intending to be bound by theory, the inventors posit that among the actives in the cosmetic composition, for example, the embodiments of the cosmetic composition as exemplified herein, the encapsulated retinol regulates cell proliferation and increases collagen and elastin while being less irritating than free retinol, and the blend of peptides, in particular tetradecyl aminobutyroylvalylaminobutyric urea trifluoroacetate and hydrolyzed rice protein, stimulate hyaluronic acid production and increase proteoglycans, and the glaucine reduces metalloproteinases and adipocyte differentiation. Independent of the specific mechanisms and any synergy between these actives, the results obtained with the exemplified embodiments of the inventive composition, as described in the examples, include statistically significant improvements in many signs of neck skin aging such as lines, wrinkles, crepiness, sagging, the benefits conferred by one or more of increasing firmness, increasing elasticity, improving hydration, and reducing the presence superficial adipose tissue.

In accordance with the various embodiments, the cosmetic composition comprises the following ingredients and optional additional ingredients.

### COMPOSITIONS

In accordance with the disclosure, provided are embodiments of an anti-aging cosmetic composition that include a blend of skin actives comprising at least the combination of glaucine, retinol and a blend of peptides. The blend of skin actives is provided in an aqueous carrier system, and may be in any form such as, but not limited to, a single phase, biphase, or emulsion, wherein the cosmetic composition is a leave on treatment in a product form selected from, but not limited to, a cream, gel, lotion, toner, serum, or combination of these. The cosmetic composition provides one or more benefits, including, but not limited to improvement in many signs of skin aging, particularly on the neck, such as lines, wrinkles, crepiness, and sagging, the benefits conferred by one or more of increasing firmness, increasing elasticity, improving hydration, and reducing the presence superficial adipose tissue.

The cosmetic composition may include one or more of additional skin actives to provide one or more additional benefits, including, but not limited to brightening/whitening, blemish control or reduction, reduction of redness, and hydration.

And, the cosmetic composition may include additional ingredients including one or more of each of solvents, surfactants, oils, thickeners, preservatives, brightening agents, effect pigments, essential oils, plant extracts, or combinations of these.

Glaucine

In accordance with the various embodiments, the cosmetic composition includes glaucine.

The cosmetic composition comprises from about 0.001% to about 0.1% of glaucine, or from about 0.0025% to about 0.005%, or any suitable combination, sub-combination, range, or sub-range thereof by weight, based on the weight of the cosmetic composition. One of ordinary skill in the art, however, will appreciate that other ranges are within the scope of the invention.

Thus, glaucine is present by weight, based on the total weight of the cosmetic composition, from about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09,% to about 0.1 percent, including increments and ranges therein and there between.

Retinol

In accordance with the various embodiments, the cosmetic composition includes retinol. In some embodiments, the retinol is an encapsulated retinol. More generally, retinol, as used herein, refers to the chemical that is also known as a form of Vitamin A. Topically applied retinol is known to increase cutaneous collagen production in both photo-aged and naturally aged skin. The combination of collagen stimulation and repression of its degradation provides broad anti-aging skin benefits including fine line and wrinkle reduction. Despite the many benefits of retinol for skin, there are several drawbacks that make formulation with retinol a challenging task. Retinol is an inherently unstable molecule and it is sensitive to light and air (oxygen) which can cause isomerization of the all-trans structure and oxidation to form multiple byproducts that are less effective on skin. One strategy to help stabilize retinol is encapsulation to help protect from light and oxygen. This type of encapsulated technology has the additional advantage of controlled delivery. As the formula is applied to skin, a combination of friction and diffusion releases the retinol in a gradual manner. The encapsulation of retinol thus prevents the full amount of retinol from being immediately dosed to the skin, reducing irritation to the sensitive skin of the neck. More info on retinol encapsulation technology can be found at URL mineralstech.com/business-segments/performance-materials/household-personal-care/products/skin-care/delivery-systems/poly-pore-technology.

The cosmetic composition comprises from about 0.05% to about 0.5% of retinol, or from about 0.1% to about 0.2%, or any suitable combination, sub-combination, range, or sub-range thereof by weight, based on the weight of the cosmetic composition. One of ordinary skill in the art, however, will appreciate that other ranges are within the scope of the invention.

Thus, retinol is present by weight, based on the total weight of the cosmetic composition, from about 0.05, 0.06, 0.07, 0.08, 0.09, 0.1, 0.2, 0.3, 0.4, to about 0.5 percent, including increments and ranges therein and there between.

Peptides

In accordance with the various embodiments, the cosmetic composition includes a blend of peptides. In some embodiments, the cosmetic composition includes a blend of peptides comprising one or more of peptides selected from di-peptides, tri-peptides and combinations thereof. In some embodiments, one or more amino acids in each peptide is a derivative of a natural amino acid.

In some particular embodiments, the cosmetic composition comprises a blend of peptides comprising tetradecyl aminobutyroylvalylaminobutyric urea trifluoroacetate and hydrolyzed rice protein.

In some embodiments, the peptides may include, but are not limited to, acetyl dipeptide-1 cetyl ester, palmitoyl tripeptide-1, acetyl tetrapeptide-9, palmitoyl tetrapeptide-7, palmitoyl tripeptide-8, dipeptide diaminobutyroyl benzylamide diacetate, or a combination thereof.

In accordance with the various embodiments, the cosmetic composition comprises a blend of peptides present in a range from about 0.001% to about 5%, or from about 0.0025% to about 0.025%, or from about 0.025% to about 0.25%, or from about 0.0022% to about 0.00275%, or from about 0.125% to about 5%, or from about 0.125% to about 0.250%, or from about 0.125% to about 2.5%, or from about 0.125% to about 0.25%, or any suitable combination, sub-combination, range, or sub-range thereof by weight, based on the weight of the cosmetic composition. More generally, each peptide that may be present in the cosmetic composition may be present in a range from about 0.001% to about 5%, or any of the other ranges as set forth herein, including the ranges as set forth for certain specific embodiments wherein the blend of peptides comprises each of tetradecyl aminobutyroylvalylaminobutyric urea trifluoroacetate and hydrolyzed rice protein. One of ordinary skill in the art, however, will appreciate that other ranges are within the scope of the invention. In some embodiments, the cosmetic composition includes more than one peptide, including more than one peptide in addition to each of the peptides comprising tetradecyl aminobutyroylvalylaminobutyric urea trifluoroacetate and hydrolyzed rice protein, each additional peptide present in an amount as set forth herein above, wherein each different peptide may be present within one of the ranges selected from the ranges set forth herein above.

In some particular embodiments, the cosmetic composition comprises in the blend of peptides each of peptides comprising tetradecyl aminobutyroylvalylaminobutyric urea trifluoroacetate and hydrolyzed rice protein, wherein the tetradecyl aminobutyroylvalylaminobutyric urea trifluoroacetate is present in a range from about 0.001% to about 5%, or from about 0.0025% to about 0.025%, or from about 0.025% to about 0.25%, or from about 0.0022% to about 0.00275%, or any suitable combination, sub-combination, range, or sub-range thereof; and wherein the hydrolyzed rice protein is present in a range from about 0.001% to about 5%, or from about 0.125% to about 5%, or from about 0.125% to about 0.250%, or from about 0.125% to about 2.5%, or from about 0.125% to about 0.25%, or any suitable combination, sub-combination, range, or sub-range thereof by weight, based on the weight of the cosmetic composition.

Thus, each one of the peptides present in the blend of peptides, may be present by weight, based on the total weight of the cosmetic composition, from about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 2, 3, 4, to about 5 percent, including increments and ranges therein and there between

Aqueous Carrier System

Water

In accordance with the various embodiments, the cosmetic composition includes water. The cosmetic composition comprises from about 1% to about 99% of water, or from about 10% to about 95%, or from about 15% to about 90%, or from about 40% to about 80%, or from about 45% to about 65%, or from about 55% to about 70%, or about 60% or any suitable combination, sub-combination, range, or sub-range thereof by weight, based on the weight of the cosmetic composition. One of ordinary skill in the art, however, will appreciate that other ranges are within the scope of the invention.

Thus, water is present by weight, based on the total weight of the cosmetic composition, from about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95 to about 99 percent, including increments and ranges therein and there between.

The pH of the cosmetic composition is not limited but is generally greater than or equal % to about pH 6, and in some embodiments, is one of between 5 and 8, and between 5 and 7, and between 6 and 7, and in some embodiments is about 6. The pH can be adjusted to the desired value by addition of a base (organic or inorganic) to the cosmetic composition, for example ammonia or a primary, secondary or tertiary (poly)amine, such as monoethanolamine, diethanolamine, triethanolamine, isopropanolamine or 1,3-propanediamine, or alternatively by addition of an inorganic or organic acid, advantageously a carboxylic acid, such as, for example, citric acid.

Water-Soluble Solvents

In accordance with some embodiments, the cosmetic composition may include at least one water-soluble solvent. The term “water-soluble solvent” is interchangeable with the term “water-miscible solvent” and means a compound that is liquid at 25° C. and at atmospheric pressure (760 mmHg), and it has a solubility of at least 50% in water under these conditions. In some cases, the water-soluble solvent has a solubility of at least 60%, 70%, 80%, or 90% in water under these conditions. Non-limiting examples of water-soluble solvents include, for example, glycerin, alcohols (for example, C1-C30, C1-C15, C1-C10, or C1-C4 alcohols), organic solvents, polyols, glycols, or mixtures thereof.

In some particular embodiments according to the disclosure, when present, a water-soluble solvent may include caprylyl glycol, glycerin, or combinations thereof.

As examples of organic solvents, non-limiting mentions can be made of monoalcohols and polyols such as ethyl alcohol, isopropyl alcohol, propyl alcohol, benzyl alcohol, and phenylethyl alcohol, or glycols or glycol ethers such as, for example, monomethyl, monoethyl and monobutyl ethers of ethylene glycol, propylene glycol or ethers thereof such as, for example, monomethyl ether of propylene glycol, butylene glycol, hexylene glycol, dipropylene glycol as well as alkyl ethers of diethylene glycol, for example monoethyl ether or monobutyl ether of diethylene glycol. Other suitable examples of organic solvents are ethylene glycol, propylene glycol, butylene glycol, hexylene glycol, propane diol, and glycerin. The organic solvents can be volatile or non-volatile compounds.

Further non-limiting examples of water-soluble solvents include alkanols (polyhydric alcohols such as glycols and polyols) such as glycerin, 1,2,6-hexanetriol, trimethylolpropane, ethylene glycol, propylene glycol, diethylene glycol, butylene glycol, hexylene glycol, triethylene glycol, tetraethylene glycol, pentaethylene glycol, dipropylene glycol, 1,3-butanediol, 2,3-butanediol, 1,4-butanediol, 3-methyl-1,3-butanediol, 1,5-pentanediol, tetraethylene glycol, 1,6-hexanediol, 2-methyl-2,4-pentanediol, polyethylene glycol, 1,2,4-butanetriol, 1,2,6-hexanetriol, 2-butene-1,4-diol, 2-ethyl-1,3-hexanediol, 2-methyl-2,4-pentanediol, (caprylyl glycol), 1,2-hexanediol, 1,2-pentanediol, and 4-methyl-1,2-pentanediol; alkyl alcohols having 1 to 4 carbon atoms such as ethanol, methanol, butanol, propanol, and isopropanol; glycol ethers such as ethylene glycol monomethyl ether, ethylene glycol monoethyl ether, ethylene glycol monobutyl ether, ethylene glycol monomethyl ether acetate, diethylene glycol monomethyl ether, diethylene glycol monoethyl ether, diethylene glycol mono-n-propyl ether, ethylene glycol mono-iso-propyl ether, diethylene glycol mono-iso-propyl ether, ethylene glycol mono-n-butyl ether, ethylene glycol mono-t-butyl ether, diethylene glycol mono-t-butyl ether, 1-methyl-1-methoxybutanol, propylene glycol monomethyl ether, propylene glycol monoethyl ether, propylene glycol mono-t-butyl ether, propylene glycol mono-n-propyl ether, propylene glycol mono-iso-propyl ether, dipropylene glycol monomethyl ether, dipropylene glycol monoethyl ether, dipropylene glycol mono-n-propyl ether, and dipropylene glycol mono-iso-propyl ether; 2-pyrrolidone, N-methyl-2-pyrrolidone, 1,3-dimethyl-2-imidazolidinone, formamide, acetamide, dimethyl sulfoxide, sorbit, sorbitan, acetine, diacetine, triacetine, sulfolane, or mixtures thereof.

In some cases, a water-soluble solvent may be selected from the group consisting of one or more glycols, C1-4 alcohols, glycerin, or mixtures thereof.

In accordance with the various embodiments the amount of the at least one water-soluble solvent, when present, is from about 0.1% to about 25%, or from about 0.1% to about 2%, or from about 0.1% to about 1%, or from about 0.1% to about 0.8%, or from about 0.1% to about 0.5%, or from about 1% to about 20%, or from about 1% to about 10%, or from about 2% to about 8%, or any suitable combination, sub-combination, range, or sub-range thereof by weight, based on the weight of the cosmetic composition. One of ordinary skill in the art, however, will appreciate that other ranges are within the scope of the invention. In some embodiments, the cosmetic composition includes more than one water soluble solvent, each water soluble solvent present in an amount as set forth herein above, wherein each different water soluble solvent may be present within one of the ranges selected from the ranges set forth herein above.

Thus, each one or combination of water-soluble solvents, when present, may be present by weight, based on the total weight of the cosmetic composition, from about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24 to about 25 weight percent, including increments and ranges therein and there between.

Additional Ingredients

In various embodiments, the cosmetic composition may include one or more of additional skin actives, thickeners, surfactants, oils, fillers, preservatives, pigments, brightening agents, effect pigments, dyes and colorants, essential oils, plant extracts, fragrances, pH adjusters, chelating agents, such as EDTA, dispersants, powders, opacifiers, or combinations of these.

Additional Actives

In accordance with some embodiments, the cosmetic composition may comprise one or more additional actives, and in some embodiments, more than one additional active. In some embodiments, there may be one or more actives present in the cosmetic composition, according to the disclosure, the active selected to provide one or more benefits, including, but not limited to brightening/whitening, improvement of skin tone, blemish control or reduction, reduction of redness, anti-aging, and hydration.

In some embodiments, actives may be selected from anti-aging ingredients including hydroxypropyl tetrahydropyrantriol, one or more humectant, such as methyl gluceth-20, and one or more vitamin or vitamin derivative, such as one of sodium citrate, citric acid, dipotassium glycyrrhizate, 3-O-ethyl ascorbic acid, tocopherol, Anogeissus leiocarpus bark extract, hydrolyzed Rhodophycea extract, hydrolyzed linseed extract, Pseudoalteromonas ferment extract, Manilkara multinervis leaf extract, Lavandula Hybrida oil, Grifola frondosa fruiting body extract, Plantago Lanceolata leaf extract, Cyathea medullaris leaf extract, hydrolyzed hyaluronic acid, Malus Domestica fruit cell culture extract, resveratrol, Salicyloyl Phytosphingosine and combinations thereof.

In some embodiments, actives may be selected from oily skin treatment ingredients including lens esculenta (lentil) seed extract, sarcosine, Peumus boldus leaf extract, Crithmum maritimum extract, niacinamide, salicylic acid, hydroxyethylpiperazine ethane sulfonic acid, Spiraea ulmaria extract, pentaerythrityl tetra-di-t-butyl hydroxyhydrocinnamate and combinations thereof.

In some embodiments, actives may be selected from skin tone improving ingredients including Ophiopogon japonicus root extract, Chenopodium quinoa seed extract, Tamarindus indica seed gum, xylitylglucoside (and) anhydroxylitol (and) xylitol, ceramide derivatives, Helianthus annuus (sunflower) seed oil unsaponifiables, myristyl malate phosphonic acid, mannose, 2-oleamido-1,3-octadecanedi and combinations thereof.

In some embodiments, actives may be selected from anti-redness ingredients including madecassoside, saccharide isomerate, palmitoyl tripeptide-8, panthenol, Olea europaea (olive) leaf extract, Mentha Piperita (peppermint) extract, Leontopodium alpinum extract, dipotassium glycyrrhizate, acetyl dipeptide-1 cetyl ester, acetyl tetrapeptide-15, Boswellia serrata extract, sodium palmitoyl proline (and) nymphaea alba flower extract and combinations thereof.

In some embodiments, actives may be selected from glycerin and glycols such as, for example, propylene glycol, butylene glycol, dipropylene glycol and diethylene glycol, glycol ethers such as monopropylene, dipropylene and tripropylene glycol alkyl(C₁-C₄) ethers, monoethylene, diethylene and triethylene glycol; salicylic acid and combinations thereof.

In some embodiments, actives may be selected from extracts of algae and of planktons, enzymes and coenzymes, ceramides, anti-oxidant compounds, including, phenolic compounds, such as chalcones, flavones, flavanones, flavanols, flavonols, dihydroflavonols, isoflavonoids, neoflavonoids, catechins, anthocyanidins, tannins, lignans, aurones, stilbenoids, curcuminoids, alkylphenols, betacyanins, capsacinoids, hydroxybenzoketones, methoxyphenols, naphthoquinones, and phenolic terpenes, resveratrol, curcumin, pinoresinol, ferulic acid, hydroxytyrosol, cinnamic acid, caffeic acid, p-coumaric acid, baicalin, pine bark, and other extracts and combinations thereof.

In some embodiments, actives may be selected from anti-elastase and anti-collagenase agents, peptides, fatty acid derivatives, steroids; free-radical scavengers, vitamins and vitamin derivatives, such as ascorbic acid, and Vitamin E.

In some embodiments, actives may be selected from sunscreen agents (e.g., inorganic sunscreen agent, such as titanium dioxide and zinc oxide and organic sunscreen agents, such as octocrylene, ethylhexyl salicylate, homosalate, and avobenzone); and combinations thereof.

In some particular embodiments, one or more optional additional active is water soluble.

In accordance with the various embodiments, one or more additional actives, alone or in combination, when present, can be present in the cosmetic composition from about 0.00001% to about 25%, or from about 0.00005% to about 15%, or from about 0.0001% to about 2%, or from about 0.05% to about 1%, or from about 0.02% to about 2%, or from about 0.2% to about 1%, or any suitable combination, sub-combination, range, or sub-range thereof by weight, based on the weight of the cosmetic composition. One of ordinary skill in the art, however, will appreciate that other ranges are within the scope of the invention. In some embodiments, the cosmetic composition comprises two, three, four or more actives having some, or all of which may be water soluble. In some embodiments, the cosmetic composition includes more than one additional active, each additional active present in an amount as set forth herein above, wherein each different additional active may be present within one of the ranges selected from the ranges set forth herein above.

Thus, each one or combination of additional actives, when present, may be present by weight, based on the total weight of the cosmetic composition, from about 0.00001, 0.00002, 0.00003, 0.00004, 0.00005, 0.00006, 0.00007, 0.00008, 0.00009, 0.0001, 0.0002, 0.0003, 0.0004, 0.0005, 0.0006, 0.0007, 0.0008, 0.0009, 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 2, 3, 4, 5, 6, 7, 8, 9, 10, 11, 12, 13, 14, 15, 16, 17, 18, 19, 20,% to about 25 percent, including increments and ranges therein and there between.

Thickener

In accordance with some embodiments, the cosmetic composition may comprise one or more thickener. The thickener may be synthetic, mineral or organic in nature, and may comprise a particulate of any shape.

In some particular embodiments according to the disclosure, when present, a thickener may include xanthan gum.

By way of non-limiting example, thickeners may be chosen from xanthan gum, cellulose gum, acacia senegal gum, guar gum, sclerotium gum, dehydroxanthan gum, gellan gum, agar, algin, talc, mica, silica, silica surface-treated with a hydrophobic agent, fumed silica, kaolin, clay/smectite mineral (Argilla extract) polyamide (NYLON) powders (e.g. ORGASOL from Atochem), polyurethane powders, poly-β-alanine powder and polyethylene powder, powders of tetrafluoroethylene polymers (TEFLON), lauroyllysine, starch, boron nitride, hydrophobic silica aerogel particles, hollow polymer microspheres such as those of polyvinylidene chloride/acrylonitrile, for instance EXPANCEL (Nobel Industrie) or of acrylic acid copolymers (POLYTRAP from the company Dow Corning) and silicone resin microbeads (TOSPEARLS from Toshiba, for example), elastomeric polyorganosiloxane particles, precipitated calcium carbonate, magnesium carbonate, magnesium hydrogen carbonate, hydroxyapatite, hollow silica microspheres (SILICA BEADS from Maprecos), glass or ceramic microcapsules, and metal soaps derived from organic carboxylic acids containing from 8 to 22 carbon atoms and preferably from 12 to 18 carbon atoms, for example zinc stearate, magnesium stearate, lithium stearate, zinc laurate, magnesium myristate and combinations thereof.

In accordance with some embodiments, one or more thickener may be present in the cosmetic composition from about 0.01% to about 10%, or from about 0.01% to about 5%, or from about 0.01% to about 1%, or from about 0.01% to about 0.2%, or from about 0.01% to about 0.09%, or from about 0.02% to about 0.08%, or any suitable combination, sub-combination, range, or sub-range thereof by weight, based on the weight of the cosmetic composition. One of ordinary skill in the art, however, will appreciate that other ranges are within the scope of the invention. In some embodiments, the cosmetic composition includes more than one thickener, each thickener present in an amount as set forth herein above, wherein each different thickener may be present within one of the ranges selected from the ranges set forth herein above.

In at least certain exemplary embodiments, the thickener is present in an amount less than about 1%, such as less than about 0.5%, or less than about 0.1%. It will be appreciated by one of skill in the art that the amount of thickener used will provide for lesser or greater thickening of the cosmetic composition, thus enabling adjustment of thickness for a specific purpose. Thus, lesser amounts of thickener will be suitable for watery, toner type forms of the cosmetic composition.

Thus, each one or combination of additional thickeners, when present, may be present by weight, based on the total weight of the cosmetic composition, from about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 2, 3, 4, 5, 6, 7, 8, 9, to about 10 percent, including increments and ranges therein and there between.

Surfactant

In accordance with some embodiments, the cosmetic composition may include one or more surfactants. In accordance with some particular embodiments, the cosmetic composition may comprise an emulsion that comprises one or more surfactants, alone or in combination, with one or more oil (as described below).

In some particular embodiments according to the disclosure, when present, a surfactant may include glyceryl stearate (for example, glyceryl stearate SE), stearic acid, potassium cetyl phosphate, or combinations thereof.

By way of non-limiting example, surfactants may be chosen from glyceryl stearate (for example, glyceryl stearate SE), stearic acid, potassium cetyl phosphate, and combinations thereof.

The one or more surfactant in the cosmetic composition may be present in an amount ranging from about 0.1% to about 5%, or from about 0.1% to about 1%, or from about 0.2% to about 0.8%, or from about 0.3% to about 0.5%. In at least certain exemplary embodiments, the surfactant is present in an amount less than about 1%, or less than about 0.8%. In some embodiments, the cosmetic composition includes more than one surfactant, each surfactant present in an amount as set forth herein above, wherein each different surfactant may be present within one of the ranges selected from the ranges set forth herein above.

Thus, each one or combination of surfactants, when present, may be present by weight, based on the total weight of the cosmetic composition, from about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2, 3, 4, to about 5 percent, including increments and ranges therein and there between.

Oil

In accordance with some embodiments, the cosmetic composition may include one or more oils. In accordance with some particular embodiments, the cosmetic composition may comprise an emulsion that comprises one or more oils, alone or in combination, with one or more surfactant (as described above).

As used herein, oil refers to any nonpolar compound that is a liquid at 25° C. and is hydrophobic and lipophilic. Oil that is suitable for use herein may be volatile or non-volatile. The term “volatile oil” relates to oil that is capable of evaporating on contact with the skin or a keratin fiber in less than one hour, at room temperature and atmospheric pressure. The volatile oil(s) are liquid at room temperature and have a non-zero vapor pressure, at room temperature and atmospheric pressure, ranging in particular from 0.13 Pa to 40 000 Pa (10-3 to 300 mmHg). The term “non-volatile oil” relates to oil which remains on the skin or the keratin fiber, at room temperature and atmospheric pressure, for at least several hours and which in particular has a vapor pressure of less than 10-3 mmHg (0.13 Pa).

In some particular embodiments according to the disclosure, when present, an oil may include Helianthus Annuus Seed Oil Unsaponifiables, Lavandula Angustifolia Oil, Mentha Piperita Oil, Rosmarinus Officinalis Leaf Oil, Dimethicone, Hydrogenated Polyisobutene, Myristyl Myristate, or combinations thereof.

In accordance with the disclosure, by way of non-limiting example, the one or more oil in the cosmetic composition may be chosen from botanical and essential oils, such as Helianthus Annuus Seed Oil, Lavandula Angustifolia (lavender) Oil, Mentha Piperita Oil, Rosmarinus Officinalis (rosemary) Leaf Oil Pelargonium Graveolens flower oil, Citrus Aurantium dulcis (orange) peel oil, Menthe Viridis (spearmint) leaf oil, Citrus aurantifolia (lime) oil, Melaleuca alternifolia (tea tree) leaf oil, Citrus Grandis (grapefruit) peel oil, Citrus medica limonum (lemon) peel oil, rose flower oil, Eucalyptus globulus leaf oil, and combinations thereof.

The term “silicone oil” relates to oil comprising at least one silicon atom, and especially at least one Si—O group. The term “fluoro oil” relates to oil comprising at least one fluorine atom. The term “hydrocarbon-based oil” relates to oil comprising mainly hydrogen and carbon atoms. Hydrocarbon-based oil may be animal hydrocarbon-based oil, plant hydrocarbon-based oil, mineral hydrocarbon-based oil or a synthetic hydrocarbon-based oil. Further, suitable oil may be a mineral hydrocarbon-based oil, a plant hydrocarbon-based oil, or a synthetic hydrocarbon-based oil.

Silicone Oils

The cosmetic composition may comprise one or more silicone oils. Non-limiting examples of silicone oils include dimethicone, cyclomethicone, polysilicone-11, phenyl trimethicone, trimethylsilylamodimethicone, and stearoxytrimethylsilane. In some cases, the cosmetic composition includes dimethicone, and optionally additional oils, including additional silicone oils. Typically, the one or more silicone oils is a non-volatile silicon oil. In some embodiments, the silicone oil is polydimethylsiloxanes (PDMSs), polydimethylsiloxanes comprising alkyl or alkoxy groups which are pendent and/or at the end of the silicone chain, which groups each contain from 2 to 24 carbon atoms, or phenyl silicones, such as phenyl trimethicones, phenyl dimethicones, phenyl(trimethylsiloxy)diphenylsiloxanes, diphenyl dimethicones, diphenyl(methyldiphenyl)trisiloxanes or (2-phenylethyl)trimethylsiloxysilicates.

Other examples of silicone oils that may be mentioned include volatile linear or cyclic silicone oils, especially those with a viscosity 8 centistokes (8×106 m2/s) and especially containing from 2 to 7 silicon atoms, these silicones optionally comprising alkyl or alkoxy groups containing from 1 to 10 carbon atoms. As volatile silicone oils that may be used in the invention, mention may be made especially of octamethylcyclotetrasiloxane, decamethylcyclopentasiloxane, dodecamethylcyclohexasiloxane, heptamethylhexyltrisiloxa ne, hepta methyloctyltrisiloxane, hexamethyldisiloxane, octamethyltrisiloxane, decamethyltetrasiloxane and dodecamethylpentasiloxane, and mixtures thereof.

Fluoro Oils

The cosmetic composition may comprise one or more fluoro oils. For example, the one or more fluoro oil may be selected from the group consisting of perfluoromethylcyclopentane, perfluoro-1,3-dimethylcyclohexane, dodecafluoropentane, tetradecafluorohexane, bromoperfluorooctyl, nonafluoromethoxybutane, nonafluoroethoxyisobutane and 4-trifluoromethylperfluoromorpholine. Volatile fluoro oils, such as nonafluoromethoxybutane, decafluoropentane, tetradecafluorohexane, dodecafluoropentane, may also be used.

Hydrocarbon-Based Oils

The cosmetic composition may comprise one or more hydrocarbon-based oils. For example, the hydrocarbon-based oil may be a saturated hydrocarbon, an unsaturated hydrocarbon, lipids, triglycerides, a natural oil, and/or a synthetic oil. In some embodiments, the cosmetic composition may include a synthetic oil selected from the group consisting of hydrogenated polyisobutene and hydrogenated polydecene. A hydrocarbon-based oil may be a non-volatile hydrocarbon-based, such as:

(i) hydrocarbon-based oils of plant origin, such as glyceride triesters, which are generally triesters of fatty acids and of glycerol, the fatty acids of which can have varied chain lengths from C4 to C24, it being possible for these chains to be saturated or unsaturated and linear or branched; these oils are in particular wheat germ oil, sunflower oil, grape seed oil, sesame oil, corn oil, apricot oil, castor oil, rhea oil, avocado oil, olive oil, soybean oil, sweet almond oil, palm oil, rapeseed oil, cottonseed oil, hazelnut oil, macadamia oil, jojoba oil, alfalfa oil, poppy oil, pumpkin seed oil, marrow oil, blackcurrant oil, evening primrose oil, millet oil, barley oil, quinoa oil, rye oil, safflower oil, candlenut oil, passionflower oil, and musk rose oil.

(ii) synthetic ethers containing from 10 to 40 carbon atoms;

(iii) linear or branched hydrocarbons of mineral or synthetic origin, such as petroleum jelly, polydecenes, hydrogenated polyisobutene such as Parleam, and 40 squalane;

(iv) synthetic esters, for instance oils of formula RCOOR′ in which R represents a linear or branched fatty acid residue containing from 1 to 40 carbon atoms and R′ represents a hydrocarbon-based chain that is especially branched, containing from 1 to 40 carbon atoms on condition that R+R′ is ÿ 10, for instance Purcellin oil (cetearyl octanoate), isopropyl myristate, isopropyl palmitate, C12-C15 alkyl benzoate, such as the product sold under the trade name Finsolv TN® or Witconol TN® by Witco or Tegosoft TN® by Evonik Goldschmidt, 2-ethylphenyl benzoate, such as the commercial product sold under the name X-Tend 226 by ISP, isopropyl lanolate, hexyl laurate, diisopropyl adipate, isononyl isononanoate, oleyl erucate, 2-ethylhexyl palmitate, isostearyl isostearate, diisopropyl sebacate, such as the product sold under the name of “Dub Dis” by Stearinerie Dubois, octanoates, decanoates or ricinoleates of alcohols or polyalcohols, such as propylene glycol dioctanoate; hydroxylated esters, such as isostearyl lactate or diisostearyl malate; and pentaerythritol esters; citrates or tartrates, such as di(linear C12-C13 alkyl) tartrates, such as those sold under the name Cosmacol ETI® by Enichem Augusta Industriale, and also di(linear C14-C15 alkyl) tartrates, such as those sold under the name Cosmacol ETL® by the same company; or acetates;

(v) fatty alcohols that are liquid at room temperature, containing a branched and/or unsaturated carbon-based chain containing from 12 to 26 carbon atoms, for instance octyldodecanol, isostearyl alcohol, oleyl alcohol, 2-hexyldecanol, 2-butyloctanol or 2-undecylpentadecanol;

(vi) higher fatty acids, such as oleic acid, linoleic acid or linolenic acid;

(vii) carbonates, such as dicaprylyl carbonate, such as the product sold under the name Cetiol CC® by Cognis;

(viii) fatty amides, such as isopropyl N-lauroyl sarcosinate, such as the product sold under the trade name Eldew SL 205® from Ajinomoto; and

(ix) essential oils selected from the group consisting of sunflower oil, sesame oil, peppermint oil, macadamia nut oil, tea tree oil, evening primrose oil, sage oil, rosemary oil, coriander oil, thyme oil, pimento berries oil, rose oil, anise oil, balsam oil, bergamot oil, rosewood oil, cedar oil, chamomile oil, sage oil, clary sage oil, clove oil, cypress oil, eucalyptus oil, fennel oil, sea fennel oil, frankincense oil, geranium oil, ginger oil, grapefruit oil, jasmine oil, juniper oil, lavender oil, lemon oil, lemongrass oil, lime oil, mandarin oil, marjoram oil, myrrh oil, neroli oil, orange oil, patchouli oil, pepper oil, black pepper oil, petitgrain oil, pine oil, rose otto oil, rosemary oil, sandalwood oil, spearmint oil, spikenard oil, vetiver oil, wintergreen oil, and ylang ylang.

Hydrocarbon-based oils may be glyceride triesters and in particular to caprylic/capric acid triglycerides, synthetic esters and in particular isononyl isononanoate, oleyl erucate, C12-C15 alkyl benzoate, 2-ethylphenyl benzoate and fatty alcohols, such as octyldodecanol. As volatile hydrocarbon-based oils, mention is made of hydrocarbon-based oils containing from 8 to 16 carbon atoms and in particular of branched C8-C16 alkanes, such as C8-C16 isoalkanes of petroleum origin (also known as isoparaffins), such as isododecane (also known as 2,2,4,4,6-pentamethylheptane), isodecane or isohexadecane, the oils sold under the Isopar or Permethyl trade names, branched C C8-C16 esters, and isohexyl neopentanoate.

In some embodiments, the cosmetic composition may comprise one or more oils such as from those described herein above, and from oils that may be selected from branched or linear, liquid alkane with carbon chain length of C11 to C20. In various embodiments, liquid alkanes may be selected from those with a carbon chain length of from C11 to C20. The liquid alkanes may be selected from those with a carbon chain length of from C11 to C20, or from C15 to C19, or one of C11, C12, C13, C14, C15, C16, C17, C18 to C19. In some particular embodiments, suitable liquid alkanes that may be used according to the disclosure include hydrocarbon-based oils containing from 8 to 16 carbon atoms, and especially branched C8-C16 alkanes such as C8-C16 isoalkanes.

In some embodiments, the cosmetic composition may comprise one or more oils selected from polar emollients selected from esters, triglycerides, ethers, carbonates, alcohols, oils, butters, fatty acids, and their combinations thereof. In various embodiments, the polar emollients may be selected from those with a molecular weight of 400 g/mol or less. More, generally, the polar emollient may have a molecular weight in the range from about 50 g/mol % to about 350 g/mol.

In some embodiments, the cosmetic composition may comprise polar emollients that include those derived from C12-050 fatty acids, preferably C16-C22 saturated fatty acids, and monohydric alcohols. In some embodiments, such esters may be chosen from isopropyl myristate, methyl palmitate, isopropyl laurate, isopropyl palmitate, ethylhexyl palmitate, ethylhexyl laurate, ethylhexyl oleate, ethylhexyl isononanoate, myristyl myristate, 2-ethylhexyl caprate/caprylate (or octyl caprate/caprylate), 2-ethylhexyl palmitate, isostearyl neopentanoate, isononyl isononanoate, hexyl laurate, esters of lactic acid and of fatty alcohols comprising 12 or 13 carbon atoms, dicaprylyl carbonate and their mixtures.

The one or more oil, when present, may be present in the cosmetic composition from about 0.0001% to about 20% or from about 0.001% to about 0.0010%, or from about 0.003% to about 0.004%, or from about 0.01% to about 0.1%, or from about 0.1% to about 10%, or from about 0.5% to about 20%, or from about 1% to about 10%, or from about 5% to about 10%, or from about 2% to about 7%, or from about 0.5% to about 2%, or from about 0.008% to about 0.01%, or from about 0.1% to about 0.2%, or from about 0.5% to about 2%, or from about or any suitable combination, sub-combination, range, or sub-range thereof by weight, based on the weight of the cosmetic composition. One of ordinary skill in the art, however, will appreciate that other ranges are within the scope of the invention. In some embodiments, the cosmetic composition includes more than one oil, each oil present in an amount as set forth herein above, wherein each different oil (such as, for example, plant oils and extracts with oils) may be present within one of the ranges selected from the ranges set forth herein above.

Thus, each one or combination of oils, when present, may be present by weight, based on the total weight of the cosmetic composition, from about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2, 3, 4, 5, 6, 7, 8, 9 to about 10 percent, including increments and ranges therein and there between.

Fillers

In accordance with some embodiments, the cosmetic composition may comprise one or more fillers. The fillers may be of mineral or organic origin, natural or synthetic in nature in order to provide oil absorption or optical effects. Oil absorption fillers may impart a matte effect and non-greasy feeling onto the skin. Optical effects fillers may impart a soft-focus/haze/blur effect to the skin, provide the skin with a more uniform appearance, reduce the appearance of skin imperfections or discoloration, or reduce the visibility of pores.

In some particular embodiments according to the disclosure, when present, a filler may include vinyl dimethicone/methicone silsesquioxane crosspolymer, ammonium polyacryloyldimethyl taurate, methyl methacrylate crosspolymer, silica silylate, or combinations thereof.

Some examples of oil-absorbing fillers include: mica, zea may (corn) starch, magnesium oxide, nylon-12, nylon-66, cellulose, polyethylene, talc, talc (and) methicone, talc (and) dimethicone, perlite, sodium silicate, pumice, PTFE, Ammonium Polyacryloyldimethyl Taurate, polymethyl methacrylate, Oryza sativa (rice) starch, aluminum starch octenylsuccinate, potato starch modified, alumina, calcium sodium borosilicate, magnesium carbonate, hydrated silica, dimethicone/vinyl dimethicone crosspolymer, sodium carboxymethyl starch. According to one preferred embodiment, the oil-absorbing filler comprises spherical microparticles of porous silica having a mean particle size from 0.5 to 20 μm whose INCI name is silica sold by the company JCG Catalysts and Chemicals under the name Spheron L-1500. According to another preferred embodiment, the oil absorbing filler comprises hydrophobic aerogel particles whose INCI name is silica silylate sold by Dow Corning under the name VM-2270 Aerogel Fine Particles.

Some examples of optical effects fillers include: bismuth oxychloride, silica silylate, boron nitride, iron oxide, calcium carbonate, calcium sulfate (and) iron oxides, sodium potassium aluminum silicate.

Some examples of fillers which provide both oil-absorbing and optical effects include: silica, silica (and) methicone, silica (and) dimethicone, polysilicone-22, polysilicone-8, polysilicone-11, methyl methacrylate crosspolymer, polymethylsilsesquioxane, methylsilanol/silicate crosspolymer, vinyl dimethicone/methicone silsesquioxane crosspolymer, diphenyl dimethicone/vinyl diphenyl dimethicone silsesquioxane crosspolymer, and styrene/acrylates copolymer.

The filler may be present in the cosmetic composition according to the invention, at a concentration, or from about 0.01% to 25%, or from about 0.01% to 0.5%, or from about 0.1% to 0.4%, or from about 0.2% to 0.3%, or from about 0.5% to 5.0%, or from about 1.0% to 5.0%, or from about 1.2% to 3.5%, or from about 1.0% to 2.5%, or from about 1.5% to about 2%, or from about 1.0% to about 1.8%, or from about 2.2% to about 2.7% or from about 0.25% to about 0.4%, or any suitable combination, sub-combination, range, or sub-range thereof by weight, based on the weight of the cosmetic composition. One of ordinary skill in the art, however, will appreciate that other ranges are within the scope of the invention In some embodiments, the cosmetic composition includes more than one filler, each filler present in an amount as set forth herein above, wherein each different filler may be present within one of the ranges selected from the ranges set forth herein above.

Thus, each one or combination of filler, when present, may be present by weight, based on the total weight of the cosmetic composition, from about 0.01, 0.02, 0.03, 0.04, 0.05, 0.06, 0.07, 0.08, 0.09, 1.0, 1.1, 1.2, 1.3, 1.4, 1.5, 1.6, 1.7, 1.8, 1.9, 2.0, 3.0, 4.0, 5.0, 6.0, 7.0, 8.0, 9.0, 10.0, 11.0, 12.0, 13.0, 14.0, 15.0, 16.0, 17.0, 18.0, 19.0, 20.0, 21.0, 22.0, 23.0, 24.0 to about 25.0 percent by weight, including increments and ranges there between.

Optional Components

In accordance with some embodiments, the cosmetic composition may comprise one or more additional optional components present in the cosmetic composition, according to the disclosure, selected from, preservatives; fragrances; pH adjusters; chelating agents, such as EDTA; dispersants; pigments, effect pigments, dyes and colorants; powders; opacifiers; and combinations thereof. Although the aforementioned optional components are given as an example, it will be appreciated that other optional components compatible with cosmetic applications known in the art may be used.

In some particular embodiments according to the disclosure, when present, optional components may include capryloyl glycine as a preservative, sodium hydroxide, or combinations thereof.

In accordance with the various embodiments, when present, the amount of optional components present in the cosmetic composition can range from about 0.001% to about 50%, or from about 0.01% to about 30%, or from about 1.5% to about 20%, and from about 5% to about 15%, or from about 0.005% to about 0.1%, or from about 0.05% to about 1%, or from about 0.06% to about 0.075%, or from about 0.1% to about 1%, or from about 0.25% to about 5%, and from about 0.5% to about 3.5%, or from about 0.2% to about 0.8%, or any suitable combination, sub-combination, range, or sub-range thereof by weight, based on the weight of the cosmetic composition. One of ordinary skill in the art, however, will appreciate that other ranges are within the scope of the invention. In some embodiments, the cosmetic composition includes more than one optional components, each optional components present in an amount as set forth herein above, wherein each different optional components may be present within one of the ranges selected from the ranges set forth herein above.

Thus, each one or combination of optional components, when present, may be present by weight, based on the total weight of the cosmetic composition, from about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 2, 3, 4, 5, 6, 7, 8, 9, 10, 11, 12, 13, 14, 15, 16, 17, 18, 19, 20, 25, 30, 35, 40, 45 to about 50 percent, including increments and ranges therein and there between.

The following Examples are provided for illustrative purposes only and are not intended to be limiting.

### EXAMPLES

### Example: Inventive Compositions

Representative examples of inventive compositions are shown in Table 1.

TABLE 1
Inventive cosmetic composition examples:

| | Exemplary Inventive |
|---|---|
| Ingredient | Formulations |
| ALLYL METHACRYLATES CROSSPOLYMER (and) | ~0.1% to about 0.2% retinol active |
| RETINOL (and) POLYSORBATE 20 | |
| HYDROLYZED RICE PROTEIN | ~0.125% to about 0.25% active |
| GLAUCINE | ~0.0025% to about 0.0005% active |
| TETRADECYL | ~0.0022% to about 0.00275% active |
| AMINOBUTYROYLVALYLAMINOBUTYRIC UREA | |
| TRIFLUOROACETATE | |
| CAPRYLOYL GLYCINE | ~0.2% to about 0.8% |
| CAPRYLYL GLYCOL | ~ 0.1% to about 1% |
| GLYCERIN | ~5% to about 10% |
| GLYCERYL STEARATE SE, STEARIC ACID, | ~0.2% to about 0.8%, each |
| POTASSIUM CETYL PHOSPHATE | |
| MYRISTYL MYRISTATE | ~0.5% to about 2%, |
| DIMETHICONE | ~2% to about 7% |
| HYDROGENATED POLYISOBUTENE | ~2% to about 7% |
| HELIANTHUS ANNUUS SEED OIL | ~0.5% to about 2%, |
| UNSAPONIFIABLES | |
| LAVANDULA ANGUSTIFOLIA OIL | ~0.008% to about 0.01% |
| ARGILLA | ~0.1% to about 0.2%, |
| MENTHA PIPERITA OIL | ~0.003% to about 0.004% |
| ROSMARINUS OFFICINALIS LEAF OIL | ~0.008% to about 0.01% |
| METHYL METHACRYLATE CROSSPOLYMER | ~1.5% to about 2.5% |
| SODIUM HYDROXIDE | ~0.06% to about 0.075% |
| SILICA SILYLATE | ~0.25% to about 0.4% |
| AMMONIUM POLYACRYLOYLDIMETHYL | ~1.0% to about 1.8% |
| TAURATE | |
| VINYL DIMETHICONE/METHICONE | ~2.2% to about 2.7% |
| SILSESQUIOXANE CROSSPOLYMER | |
| AQUA | ~55 to about 70% |
| XANTHAN GUM | ~0.02% to about 0.08% |

In one exemplary embodiment, the cosmetic composition includes In some embodiments, glaucine is present at about 0.005%, retinol is present at about 0.2%, tetradecyl aminobutyroylvalylaminobutyric urea trifluoroacetate is present from about 0.00275% and hydrolyzed rice protein is present at about 0.25%, all percentages by weight, based on the total weight of the cosmetic composition, the exemplary cosmetic composition further including one or more water-soluble solvents comprising Caprylyl Glycol and Glycerin at about 7%, one or more thickeners comprising xanthan gum present at about 0.1%, one or more surfactants each of which is present at about 0.5% comprising glyceryl stearate, stearic acid and potassium cetyl phosphate, one or more oils comprising Helianthus Annuus Seed Oil Unsaponifiables, Lavandula Angustifolia Oil, Mentha Piperita Oil, Rosmarinus Officinalis Leaf Oil, dimethicone, hydrogenated polyisobutene, and myristyl myristate, one or more filler comprising vinyl dimethicone/methicone silsesquioxane crosspolymer, ammonium polyacryloyldimethyl taurate, methyl methacrylate crosspolymer, and silica silylate, and one or more additional ingredients comprising capryloyl glycine as a preservative, and sodium hydroxide.

### Example 2: Performance-Properties with Inventive Composition: 16 Week Clinical Study, Hydration Study, TEWL Study, and Biopsy Study

16 Week Clinical Study

A clinical study on N=50 female subjects showed statistically significant improvements in: fine lines/wrinkles, crepiness, smoothness, firmness, elasticity, skin tone evenness, and lifting when comparing the baseline (initial) evaluation to those taken after using the product for 16 weeks. Subject self-assessment also indicated a strong agreement that product treatment improved the perception of many attributes by 16 weeks, including the neck feeling and appearing firmer/tighter and more lifted. VISIA photos showed an improvement in horizontal neck lines, neck lifting, crepiness, and neck firmness. Ultrasound measurement with 7.5 MHz probe showed statistically significant decrease in the superficial adipose tissue layer.

Hydration

Hydration of treated skin was measured with a corneometer and compared to an untreated section of the skin. There were statistically significant improvements for treated skin at 15 minutes, 4 hours, and 8 hours compared to baseline and to untreated sections of skin.

TEWL

Trans-epidermal water loss (TEWL) is a measurement of skin moisture barrier integrity. Treated skin showed a statistically significant decrease (improvement) in TEWL compared to untreated site and baseline after 1 hour.

Biopsy Study

A clinical study was conducted on N=11 female subjects who used the product for 12 weeks. A surgical biopsy was collected from the neck at both baseline and week 12.

Immunohistochemical analysis of the biopsy tissue indicated a statistically significant increase in quantity of the extracellular matrix proteins elastin and lumican.

Raw Materials

Raw materials used in the exemplified compositions include, in part, those listed in Table 2.

TABLE 2
Raw Materials (partial list)

| Material | Commercial Name | % active |
|---|---|---|
| Tetradecyl | Syn-Hycan (TM) | 0.11% |
| Aminobutyroylvalylaminobutyric | | |
| Urea Trifluoroacetate | | |
| Hydrolyzed Rice Protein | Nutripeptides GR (TM) | 6.25% |
| Retinol | Poly-Pore (TM) 120 RE | 20% |
| Glaucine | Ovaliss (TM) | 0.1% |

The articles “a” and “an,” as used herein, mean one or more when applied to any feature in embodiments of the present invention described in the specification and claims. The use of “a” and “an” does not limit the meaning to a single feature unless such a limit is specifically stated. The article “the” preceding singular or plural nouns or noun phrases denotes a particular specified feature or particular specified features and may have a singular or plural connotation depending upon the context in which it is used. The adjective “any” means one, some, or all indiscriminately of whatever quantity.

“One or more,” as used herein, means one or more and thus includes individual components as well as mixtures/combinations.

The transitional terms “comprising”, “consisting essentially of” and “consisting of”, when used in the appended claims, in original and amended form, define the claim scope with respect to what unrecited additional claim elements or steps, if any, are excluded from the scope of the claim(s). The term “comprising” is intended to be inclusive or open-ended and does not exclude any additional, unrecited element, method, step or material. The term “consisting of” excludes any element, step or material other than those specified in the claim and, in the latter instance, impurities ordinary associated with the specified material(s). The term “consisting essentially of” limits the scope of a claim to the specified elements, steps or material(s) and those that do not materially affect the basic and novel characteristic(s) of the claimed invention. All materials and methods described herein that embody the present invention can, in alternate embodiments, be more specifically defined by any of the transitional terms “comprising,” “consisting essentially of,” and “consisting of.”

Other than in the operating examples, or where otherwise indicated, all numbers expressing quantities of ingredients and/or reaction conditions are to be understood as being modified in all instances by the term “about,” meaning within 10% of the indicated number (e.g. “about 10%” means 9-11% and “about 2%” means 1.8-2.2%).

All percentages and ratios are calculated by weight unless otherwise indicated. All percentages are calculated based on the total composition unless otherwise indicated. Generally, unless otherwise expressly stated herein, “weight” or “amount” as used herein with respect to the percent amount of an ingredient refers to the amount of the raw material comprising the ingredient, wherein the raw material may be described herein to comprise less than and up to 100% activity of the ingredient. Therefore, weight percent of an active in a composition is represented as the amount of raw material containing the active that is used, and may or may not reflect the final percentage of the active, wherein the final percentage of the active is dependent on the weight percent of active in the raw material.

All ranges and amounts given herein are intended to include subranges and amounts using any disclosed point as an end point. Thus, a range of “1 to 10%, such as 2 to 8%, such as 3 to 5%,” is intended to encompass ranges of “1 to 8%,” “1 to 5%,” “2 to 10%,” and so on. All numbers, amounts, ranges, etc., are intended to be modified by the term “about,” whether or not so expressly stated. Similarly, a range given of “about 1 to 10%” is intended to have the term “about” modifying both the 1% and the 10% endpoints. Further, it is understood that when an amount of a component is given, it is intended to signify the amount of the active material unless otherwise specifically stated.

Notwithstanding that the numerical ranges and parameters setting forth the broad scope of the disclosure are approximations, unless otherwise indicated the numerical values set forth in the specific examples are reported as precisely as possible. Any numerical value, however, inherently contains certain errors necessarily resulting from the standard deviation found in their respective testing measurements. The example that follows serves to illustrate embodiments of the present disclosure without, however, being limiting in nature.

While the invention has been described with reference to one or more embodiments, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention is not limited to the particular embodiment disclosed as the best mode contemplated for carrying out this invention, but that the invention will include all embodiments falling within the scope of the appended claims. In addition, all numerical values identified in the detailed description shall be interpreted as though the precise and approximate values are both expressly identified.

What is claimed is:

2. A cosmetic composition comprising:
a. a blend of skin actives comprising at least a glaucine, retinol, and a blend of peptides comprising tetradecyl aminobutyroylvalylaminobutyric urea trifluoroacetate and hydrolyzed rice protein; and
b. an aqueous carrier system comprising water, and
wherein the cosmetic composition increases firmness, increases elasticity, increases hydration, reduces the presence superficial adipose tissue, or combinations thereof.

3. The cosmetic composition according to claim 1, wherein glaucine is present from about 0.001% to about 0.1%, retinol is present from about 0.05% to about 0.5%, tetradecyl aminobutyroylvalylaminobutyric urea trifluoroacetate is present from about 0.002% to about 5% and hydrolyzed rice protein is present from about 0.125% to about 5%, all percentages by weight, based on the total weight of the cosmetic composition.

4. The cosmetic composition according to claim 1, wherein glaucine is present from about 0.0025% to about 0.0005%, retinol is present from about 0.1% to about 0.2%, tetradecyl aminobutyroylvalylaminobutyric urea trifluoroacetate is present from about 0.0022% to about 0.00275% and hydrolyzed rice protein is present from about 0.125% to about 0.25%, all percentages by weight, based on the total weight of the cosmetic composition.

5. The cosmetic composition according to claim 1, wherein glaucine is present at about 0.005%, retinol is present at about 0.2%, tetradecyl aminobutyroylvalylaminobutyric urea trifluoroacetate is present from about 0.00275% and hydrolyzed rice protein is present at about 0.25%, all percentages by weight, based on the total weight of the cosmetic composition.

6. The cosmetic composition according to claim 1, wherein the blend of peptides is present from about 0.002% to about 5%.

7. The cosmetic composition according to claim 1, wherein the retinol is an encapsulated form of retinol.

8. The cosmetic composition according to claim 1, wherein the skin actives are provided in an aqueous carrier in a single phase, biphase or emulsion wherein the cosmetic composition is a leave on treatment in the form of a cream, gel, lotion, toner, or serum.

9. The cosmetic composition according to claim 1, wherein the cosmetic composition includes one or more of additional skin actives, solvents, surfactants, oils, thickeners, preservatives, brightening agents, effect pigments, essential oils, plant extracts, or combinations thereof.

10. The cosmetic composition according to claim 1, wherein the cosmetic composition comprises one or more of:
a. one or more water-soluble solvents comprising Caprylyl Glycol and Glycerin,
b. one or more thickeners comprising xanthan gum,
c. one or more surfactants comprising glyceryl stearate, stearic acid and potassium cetyl phosphate,
d. one or more oils comprising Helianthus Annuus Seed Oil Unsaponifiables, Lavandula Angustifolia Oil, Mentha Piperita Oil, Rosmarinus Officinalis Leaf Oil, Dimethicone, Hydrogenated Polyisobutene, and Myristyl Myristate,
e. one or more filler comprising vinyl dimethicone/methicone silsesquioxane crosspolymer, Ammonium Polyacryloyldimethyl Taurate, Methyl Methacrylate Crosspolymer, and silica silylate, and
f. one or more additional ingredients comprising Capryloyl Glycine as a preservative, and Sodium Hydroxide, or
g. combinations thereof.

11. A cosmetic composition comprising:
a. a blend of skin actives comprising at least a glaucine present from about 0.0025% to about 0.005% by weight, based on the total weight of the cosmetic composition, retinol, and a blend of peptides; and
b. an aqueous carrier system comprising water, and
wherein the cosmetic composition increases firmness, increases elasticity, increases hydration, reduces the presence superficial adipose tissue, or combinations thereof.

12. The cosmetic composition according to claim 11, wherein the blend of peptides is present from about 0.002% to about 5% by weight, based on the total weight of the cosmetic composition.

13. The cosmetic composition according to claim 11, wherein retinol is present from about 0.05% to about 0.5% by weight, based on the total weight of the cosmetic composition.

14. The cosmetic composition according to claim 11, wherein retinol is present from about 0.1% to about 0.2% by weight, based on the total weight of the cosmetic composition.

15. The cosmetic composition according to claim 11, wherein the blend of peptides comprises tetradecyl aminobutyroylvalylaminobutyric urea trifluoroacetate and hydrolyzed rice protein.

16. The cosmetic composition according to claim 15, wherein the tetradecyl aminobutyroylvalylaminobutyric urea trifluoroacetate is present from about 0.002% to about 5% and the hydrolyzed rice protein is present from about 0.125% to about 5%, all percentages by weight, based on the total weight of the cosmetic composition.

17. The cosmetic composition according to claim 15, wherein the tetradecyl aminobutyroylvalylaminobutyric urea trifluoroacetate is present from about 0.0022% to about 0.00275% and the hydrolyzed rice protein is present from about 0.125% to about 0.25%, all percentages by weight, based on the total weight of the cosmetic composition.

18. The cosmetic composition according to claim 11, wherein the skin actives are provided in an aqueous carrier in a single phase, biphase or emulsion wherein the cosmetic composition is a leave on treatment in the form of a cream, gel, lotion, toner, or serum.

19. The cosmetic composition according to claim 11, wherein the cosmetic composition includes one or more of additional skin actives, solvents, surfactants, oils, thickeners, preservatives, brightening agents, effect pigments, essential oils, plant extracts, or combinations thereof.

20. A cosmetic composition comprising:
a. a blend of skin actives comprising at least a glaucine, retinol, and a blend of peptides comprising tetradecyl aminobutyroylvalylaminobutyric urea trifluoroacetate and hydrolyzed rice protein; and
b. an aqueous carrier system comprising water,
wherein glaucine is present from about 0.0025% to about 0.005%, retinol is present from about 0.1% to about 0.2%, tetradecyl aminobutyroylvalylaminobutyric urea trifluoroacetate is present from about 0.0022% to about 0.00275% and hydrolyzed rice protein is present from about 0.125% to about 0.25%, all percentages by weight, based on the total weight of the cosmetic composition, and
wherein the aqueous carrier system comprises at least one water-soluble solvent, at least one oil, at least one surfactant, at least one thickener, at least one filler, or combinations thereof, and further comprises one or more of additional skin actives, preservatives, brightening agents, effect pigments, essential oils, plant extracts, or combinations thereof, and
wherein the cosmetic composition is provided as an emulsion and is a leave on treatment in the form of a cream, gel, lotion, toner, or serum, and
wherein the cosmetic composition increases firmness, increases elasticity, increases hydration, reduces the presence superficial adipose tissue, or combinations thereof.

21. The cosmetic composition according to claim 20, wherein the cosmetic composition comprises water-soluble solvent comprising Caprylyl Glycol and Glycerin, at least one thickener comprising xanthan gum, surfactant comprising glyceryl stearate, stearic acid and potassium cetyl phosphate, oil comprising Helianthus Annuus Seed Oil Unsaponifiables, Lavandula Angustifolia Oil, Mentha Piperita Oil, Rosmarinus Officinalis Leaf Oil, Dimethicone, Hydrogenated Polyisobutene, and Myristyl Myristate, filler comprising vinyl dimethicone/methicone silsesquioxane crosspolymer, Ammonium Polyacryloyldimethyl Taurate, Methyl Methacrylate Crosspolymer, and silica silylate, and additional ingredients comprising Capryloyl Glycine as a preservative, and Sodium Hydroxide.
